(19) United States
(12) Patent Application Publication
(10) Pub. No.: US 2022/0315976 A1
(43) Pub. Date: Oct. 6, 2022

(54) METHOD FOR ANTIBIOTIC SUSCEPTIBILITY TESTING OF BLOOD SAMPLES

(71) Applicants: MICROBEDX INC., Pacific Palisades, CA (US); THE REGENTS OF THE UNIVERSITY OF CALIFORNIA, Oakland, CA (US)

(72) Inventors: Colin Wynn Halford, Los Angeles, CA (US); Scott Adam Churchman, Santa Monica, CA (US); Richard Janeczko, Glendale, CA (US)

(21) Appl. No.: 17/630,987

(22) PCT Filed: Jul. 28, 2020
(86) PCT No.: PCT/US2020/043830

§ 371 Date: Jan. 28, 2022

Related U.S. Application Data
(60) Provisional application No. 62/879,709, filed on Jul. 29, 2019.

(51) Int. Cl. C12Q 1/18; C12N 1/20; C12Q 1/689; C12N 1/06

(52) U.S. Cl. CPC C12Q 1/18; C12N 1/20; C12Q 1/689; C12N 1/06

(57) ABSTRACT

The present disclosure relates to a method for determining the susceptibility of a microorganism to an antimicrobial agent. In particular, to a method for testing the antimicrobial susceptibility of blood samples in patients with a suspected bloodstream infection. In one aspect, for example, the method comprises the steps of: (a) incubating a first blood culture derived from the blood sample; (b) terminating the incubation of the first blood culture after a period of up to about 20 hours from commencement of Step (a) to produce an incubated culture; and (c) performing a molecular phenotypic antimicrobial susceptibility test (AST) assay on the incubated culture to determine the antimicrobial susceptibility of a microorganism.

### CROSS-REFERENCE TO RELATED APPLICATION

This application claims the benefit of earlier filed U.S. provisional application No. 62/879,709 entitled Method for Antibiotic Susceptibility Testing of Blood Samples and filed Jul. 29, 2019, the entirety of which is incorporated herein by reference.

### FIELD

The present disclosure relates to a method for determining the susceptibility of a microorganism to an antimicrobial agent. In particular, the present disclosure relates to a method for testing the antimicrobial susceptibility of blood samples in patients with a suspected bloodstream infection.

### BACKGROUND

Methods for bacterial identification (ID) and antimicrobial susceptibility testing (AST) of blood samples are known generally in the art. (See. e.g., Charnot-Katsikas, Angella et al. “Use of the Accelerate Pheno System for Identification and Antimicrobial Susceptibility Testing of Pathogens in Positive Blood Cultures and Impact on Time to Results and Workflow”, Vol. 56(1), J. Clinical Microbiology vol. 56,1 e01166-17 (December 2017); Opota, O. et al. “Blood culture-based diagnosis of bacteraemia: state of the art”, Vol. 21 (4) Clinical Microbiology and Infection, 313-22 (April 2015)).

Conventional clinical bacteria ID and AST of blood samples involves first adding a patient's blood to a culture bottle either directly from the patient or from a blood collection tube. This culture bottle is then incubated in a specific incubator and is continuously monitored until the blood culture generates a detectable positive indication for the presence of a microorganism, often referred to as “blood culture positivity.” In conventional blood culture analysis methods, blood culture positivity may be detected by tracking carbon dioxide (CO₂) production within the incubated blood culture. The time it takes for a blood culture to generate a detectable blood culture positivity is often referred to as the “Time to Positivity” or “TTP”. The TTP for certain microorganisms can vary from an average of about 15 hours for the fastest growing cultures to an average of about 5 days for the most fastidious organisms. (See, e.g., Fen Pan, Wantong Zhao, and Hong Zhang, “Value of Time to Positivity of Blood Culture in Children with Bloodstream Infections,” Canadian Journal of Infectious Diseases and Medical Microbiology, vol. 2019, Article ID 5975837).

Once blood culture positivity is reached, Gram staining must be performed to confirm the presence of a microorganism in the blood sample and to determine the subsequent testing pathway. Once the presence of a microorganism is confirmed, the positive blood culture can then be sub-cultured onto agar plates and an identification assay can be performed by different methods (e.g., MALDI-TOF mass spectrometry and/or nucleic-acid based methods such as polymerase chain reaction (PCR) and gene sequencing) in as little as 6 hours or as long as 48 hours depending on the time to colony formation. Once colonies are formed, they can be used to inoculate an AST assay, typically performed by broth microdilution, either manual or automated. The standard incubation period of bacteria and antibiotics for broth microdilution is 18-20 hours. Some commercially available products generate equivalent performance data in as few as 8 hours.

Accordingly, conventional methods for bacterial identification and antimicrobial susceptibility testing of blood samples are can take anywhere from many hours to days.

Due to the high morbidity and mortality associated with many bloodstream infections, it is important that treatment for such conditions not be significantly delayed. Accordingly, when bloodstream infections are suspected in patients, antibiotic treatment is frequently implemented as the blood culturing is started, well before microorganism identification and antimicrobial susceptibility testing using conventional blood culture methods can be carried out. This often results in the administration of antibiotics, or other antimicrobial agents to a patient, without first knowing if the particular microorganism is susceptible or resistant to the particular antibiotic being administered. If the bacteria are in fact resistant, the initial course of antibiotics may be ineffective, which may contribute to a known problem/trend of patients receiving unnecessary or less effective antibiotics when other, potentially more effective antibiotics may have been available for use. This can be particularly problematic due to the rise in drug-resistant microorganisms.

Despite the advances made to date in determining the antimicrobial susceptibility of bacterial pathogens in a blood samples, there is room for improvement to address the above-mentioned problems and shortcomings of the prior art.

### SUMMARY

This summary is intended to introduce the reader to the more detailed description that follows and not to limit or define any claimed or as yet unclaimed invention. One or more inventions may reside in any combination or sub-combination of the elements or process steps disclosed in any part of this document including its claims and figures.

It is an object of the present disclosure to obviate or mitigate at least one of the above-mentioned disadvantages of the prior art.

It is another object of the present disclosure to provide a novel method for determining the antimicrobial susceptibility of a microorganism in a blood sample.

It is another object of the present disclosure to provide a novel method for antimicrobial susceptibly testing on pre-positive blood cultures.

It is another object of the present disclosure to provide a novel method for simultaneously identifying a microorganism and conducting antimicrobial susceptibly testing on pre-positive blood cultures.

It is another object of the present disclosure to provide a novel method for rapid antibiotic susceptibility testing in patients with suspected bloodstream infections.

It is another object of the present disclosure to provide a novel method treating patients with suspected bloodstream infections

Accordingly, in one of its aspects, the present disclosure provides a method for determining the antimicrobial susceptibility of a microorganism in a blood sample, the method comprising the steps of:
(a) incubating a first blood culture derived from the blood sample;
(b) terminating the incubation of the first blood culture after a period of up to about 20 hours from commencement of Step (a) to produce an incubated culture; and
(c) performing a molecular phenotypic antimicrobial susceptibility test (AST) assay on the incubated culture to determine the antimicrobial susceptibility of a microorganism.

Accordingly, in one of its aspects, the present disclosure provides a method for determining the antimicrobial susceptibility of a microorganism in a blood sample, the method comprising the steps of:
(a) incubating a first blood culture derived from the blood sample;
(b) terminating the incubation of the first blood culture after a period of up to about 15 hours from commencement of Step (a) to produce an incubated culture; and
(c) performing a molecular phenotypic antimicrobial susceptibility test (AST) assay on the incubated culture to determine the antimicrobial susceptibility of a microorganism.

Accordingly, in one of its aspects, the present disclosure provides a method for determining the antimicrobial susceptibility of a microorganism in a blood sample, the method comprising the steps of:
(a) incubating a first blood culture derived from the blood sample;
(b) terminating the incubation of the first blood culture after a period of up to about 12 hours from commencement of Step (a) to produce an incubated culture; and
(c) performing a molecular phenotypic antimicrobial susceptibility test (AST) assay on the incubated culture to determine the antimicrobial susceptibility of a microorganism.

Accordingly, in one of its aspects, the present disclosure provides a method for determining the antimicrobial susceptibility of a microorganism in a blood sample, the method comprising the steps of:
(a) incubating a first blood culture derived from the blood sample;
(b) terminating the incubation of the first blood culture after a period of up to about 10 hours from commencement of Step (a) to produce an incubated culture; and
(c) performing a molecular phenotypic antimicrobial susceptibility test (AST) assay on the incubated culture to determine the antimicrobial susceptibility of a microorganism.

Accordingly, in one of its aspects, the present disclosure provides a method for determining the antimicrobial susceptibility of a microorganism in a blood sample, the method comprising the steps of:
(a) incubating a first blood culture derived from the blood sample;
(b) terminating the incubation of the first blood culture after a period of up to about 8 hours from commencement of Step (a) to produce an incubated culture; and
(c) performing a molecular phenotypic antimicrobial susceptibility test (AST) assay on the incubated culture to determine the antimicrobial susceptibility of a microorganism.

Accordingly, in one of its aspects, the present disclosure provides a method for determining the antimicrobial susceptibility of a microorganism in a blood sample, the method comprising the steps of:
(a) incubating a first blood culture derived from the blood sample;
(b) terminating the incubation of the first blood culture after a period of up to about 6 hours from commencement of Step (a) to produce an incubated culture; and
(c) performing a molecular phenotypic antimicrobial susceptibility test (AST) assay on the incubated culture to determine the antimicrobial susceptibility of a microorganism.

Accordingly, in one of its aspects, the present disclosure provides a method for determining the antimicrobial susceptibility of a microorganism in a blood sample, the method comprising the steps of:
(a) incubating a first blood culture derived from the blood sample;
(b) terminating the incubation of the first blood culture after a period of up to about 3 hours from commencement of Step (a) to produce an incubated culture; and
(c) performing a molecular phenotypic antimicrobial susceptibility test (AST) assay on the incubated culture to determine the antimicrobial susceptibility of a microorganism.

In another of its aspects, the present disclosure provides a method for treating a bloodstream infection in a patient, the method comprising the steps of:
(a) obtaining a blood sample from a patient;
(b) incubating a first blood culture derived from the blood sample;
(c) terminating the incubation of the first blood culture after a period of up to about 20 hours from commencement of Step (b) to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

In another of its aspects, the present disclosure provides a method for treating a bloodstream infection in a patient, the method comprising the steps of:
(a) obtaining a blood sample from a patient;
(b) incubating a first blood culture derived from the blood sample;
(c) terminating the incubation of the first blood culture after a period of up to about 15 hours from commencement of Step (b) to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

In another of its aspects, the present disclosure provides a method for treating a bloodstream infection in a patient, the method comprising the steps of:
(a) obtaining a blood sample from a patient;
(b) incubating a first blood culture derived from the blood sample;
(c) terminating the incubation of the first blood culture after a period of up to about 12 hours from commencement of Step (b) to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

In another of its aspects, the present disclosure provides a method for treating a bloodstream infection in a patient, the method comprising the steps of:
(a) obtaining a blood sample from a patient;
(b) incubating a first blood culture derived from the blood sample;
(c) terminating the incubation of the first blood culture after a period of up to about 10 hours from commencement of Step (b) to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

In another of its aspects, the present disclosure provides a method for treating a bloodstream infection in a patient, the method comprising the steps of:
(a) obtaining a blood sample from a patient;
(b) incubating a first blood culture derived from the blood sample;
(c) terminating the incubation of the first blood culture after a period of up to about 8 hours from commencement of Step (b) to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

In another of its aspects, the present disclosure provides a method for treating a bloodstream infection in a patient, the method comprising the steps of:
(a) obtaining a blood sample from a patient;
(b) incubating a first blood culture derived from the blood sample;
(c) terminating the incubation of the first blood culture after a period of up to about 6 hours from commencement of Step (b) to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

In another of its aspects, the present disclosure provides a method for treating a bloodstream infection in a patient, the method comprising the steps of:
(a) obtaining a blood sample from a patient;
(b) incubating a first blood culture derived from the blood sample;
(c) terminating the incubation of the first blood culture after a period of up to about 3 hours from commencement of Step (b) to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

In certain aspects, the incubated culture has a bacterial concentration of at least about 1×10⁴ colony-forming units per milliliter (CFU/mL), about 5×10⁴ CFU/mL, about 1×10⁵ CFU/mL, or about 5×10⁵ CFU/mL.

Accordingly, as described herein below, the present inventors have developed a method for testing blood cultures for antimicrobial susceptibility without the need to wait for the TTP or identification of a microorganism in the blood cultures. This method is based on three criteria: (1) the ability to start an antibiotic susceptibility testing (AST) assay earlier in the blood culture timeline compared to current conventional practice; (2) the ability to grow bacteria more quickly during the AST assay; and (3) the use of a molecular phenotypic AST assay that generates results much faster than current conventional phenotypic tests.

In a preferred embodiment of this method, a patient blood sample is divided into at least two parts, each of which is inoculated into separate blood culture bottles (e.g., a first culture bottle and a second “reference” culture bottle). The blood culture bottles are then subjected to incubation. At some time prior to the time it would take the blood culture sample to show positive results for the presence of a microorganism in a conventional blood culture bacterial identification method (“Time to Positivity” or “TTP”), the first culture bottle is removed from incubation, creating a so-called pre-positive culture, and is sampled using a rapid AST assay. The second blood culture bottle may continue incubation according to the standard protocol and may serve as reference value for pre-positive culture.

The rapid AST assays disclosed herein use ribosomal RNA (rRNA) as a surrogate for the growth of bacteria in the AST and the response of the bacteria to the various antibiotics. rRNA may also be used to identify and differentiate individual microorganism species present in a blood sample based on detection of specific rRNA regions. This permits simultaneous performance of antibiotic susceptibility testing and bacterial identification assays on the same blood sample allowing pairing of antibiotic effects with bacterial species (or genus) present in polymicrobial infections.

Unlike current conventional blood culture analysis that can take as long as 40 to 72 hours, the assay methods described herein may generate results in as little as 3 hours, including about 2 hours of antibiotic incubation. Accordingly, the assay methods disclosed herein may detect antibiotic resistant phenotypes in a significant proportion of bloodstream infection patients in less than 8 hours from patient sampling—i.e., a single shift turn-around time.

These and other aspects will become apparent to those of skill in the art upon reviewing the present specification.

### DRAWINGS

Embodiments of the present disclosure will be described with reference to the accompanying drawing, in which:

FIG. 1, in a flowchart, illustrates the steps and timing in a preferred embodiment for determining the antimicrobial susceptibility of a microorganism in one or more blood samples;

FIG. 2 is a schematic representation of one example of a centrifugal disc containing an incubation chamber;

FIG. 3 is a cross-sectional view of a portion of the centrifugal disc of FIG. 2, taken along line 3-3; and

FIG. 4 is a schematic representation of one example of a centrifugal disc containing multiple incubation chambers.

### DESCRIPTION

Various methods and other details will be described below to provide an example of an embodiment of each claimed invention. No embodiment described below limits any claimed invention and any claimed invention may cover a method, a process and/or an apparatus that differs from those described below. The claimed inventions are not to be limited to the embodiments described below. Any invention disclosed in below that is not claimed in this patent specification may be the subject matter of another protective instrument, for example, a continuing patent application.

The term “microorganism” used herein may include, prokaryotic cells such as bacteria, fungal cells such as yeast or viruses.

The term “about” when accompanying a numerical value, is to be construed as indicating a deviation as would be appreciated by one of ordinary skill in the art to operate satisfactorily for an intended purpose.

The term “bacteria” used herein refers to any species of bacteria, including but not limited to Gram-negative and Gram-positive bacteria, anaerobic bacteria, and parasites. In certain embodiments, the bacteria may be Gram-negative bacteria, Gram-positive bacteria, or a mixture thereof. Examples of Gram-negative bacteria may include, but are not limited to Escherichia coli, Salmonella spp, Shigella spp, Enterobacteriaceae, Pseudomonas spp, Moraxella spp, Helicobacter spp, Strenotrophomonas spp, Bdellovibrio spp, and Legionella spp. Examples of Gram-positive bacteria may include, but are not limited to Enterococcus spp, Staphylococcus spp, Streptococcus spp, Actinomyces spp, Bacillus spp, Clostridium spp, Corynebacterium spp, Listeria spp, and Lactobacillus spp.

The term “blood sample” as used herein, refers to a blood sample which may be collected and stored by any means, including in a sterile container. A blood sample may be provided by or taken from any mammal, including but not limited to humans, dogs, cats, murines, simians, farm animals, sport animals and companion animals.

The term “blood culture” as used herein, refers to blood sample, or a portion thereof, that has been introduced into a culture medium.

The term “control portion” used herein refers to a portion of the blood sample which will not be exposed to an antimicrobial agent. In some embodiments, the control portion may include a series of portions of the blood sample which will not be exposed to an antimicrobial agent.

The term “test portion” used herein refers to a portion of the blood sample which is to be exposed to at least one antimicrobial agent. In some embodiments, the test portion may include a series of portions of the blood sample which are to be exposed to at least one antimicrobial agent. In some embodiments, the test portion may include 1, 2, 3, 4, 5, 6, 7, 8, 9, 10, 11, 12, 13, 14, 15 or more portions of the blood sample which are to be exposed to at least one antimicrobial agent, preferably in parallel. In some embodiments, a single test portion is exposed to one antimicrobial agent.

The term “inoculating” used herein refers to the introduction of a blood sample, or a portion thereof, to a culture medium. Once a blood sample, or a portion thereof, has been introduced into a culture medium, it may also be referred to as “an inoculate”.

The term “rRNA” used herein refers to the ribosomal ribonucleic acid of microbes present in the blood sample.

The term “Time to Positivity” or “TTP” used herein refers to the time that it would take for a blood sample to positively display the presence of a microorganism using a current conventional blood culturing detection methods.

The term “pre-positive culture” used herein refers to a blood culture for which incubation is terminated prior to the generation of a detectable positive indication for the presence of a microorganism.

The term “bloodstream infections” used herein refers to infectious diseases caused by the presence of viable bacterial or fungal microorganisms in the bloodstream (e.g., sepsis).

In certain circumstances, it is important to detect and identify microorganisms in a blood sample and to determine the antimicrobial susceptibility of such microorganisms in the diagnosis and/or treatment of bloodstream infections. Accurately detecting and identifying the presence of microorganisms in a blood sample and determining antimicrobial susceptibility thereof, may facilitate an appropriate and more effective course of treatment.

Often, the goal of such testing is to address potential drug resistance of common microorganisms, and to assure susceptibility of the microorganisms to drugs of choice for particular infections. Information obtained in microbial identification and antibiotic susceptibility testing may help clinicians prescribe the appropriate effective antibiotics or other treatment regimes. Timely detection and identification of the particular species of microorganism in a blood sample, in combination with antimicrobial susceptibility testing results, may also assist clinicians in prescribing a targeted antibiotic or other treatment, to reduce the risk of over-prescription and evolution of drug-resistance microbes. Such methods of identification may also be used by public health officials to address the growing concern of drug-resistant infections and epidemics.

In a clinical setting, a blood sample may be obtained from a patient, whether it be a human or animal, who may require further medical treatment based on the results of the analysis of the blood sample. For example, blood samples are often obtained from patients experiencing symptoms consistent with bloodstream infections (e.g., sepsis).

However, conventional methods of testing blood samples for bacterial identification (ID) and antimicrobial susceptibility testing (AST) are known to be very time intensive. For example, a conventional method for collecting a blood sample and identifying bacteria and conducting antimicrobial susceptibility testing requires culturing the blood sample and subjecting the cultured blood sample to long incubation periods, gram staining, and sub-culturing, followed by an ID and AST assay. Such methods may be relatively accurate but are relatively slow, taking many hours, days or weeks to provide useful and reliable results to the clinician. Bloodstream infections (e.g., sepsis) are associated with high morbidity and mortality, which can often be attributed to this significant delay in diagnosis and effective treatment. Consequently, in a clinical environment, these time frames may be undesirable and may be considered too long a time period to withhold/delay treatment for the patient.

Accordingly, this time delay leads to treatments being implemented, such as a particular antibiotic being prescribed to a patient, before the results from a microbial identification screen are obtained. This can sometimes lead to the unnecessary prescription of antibiotics and/or the prescription of a selected antibiotic that is less effective in treating a particular class of microorganisms than other available antibiotics.

There remains a need for relatively faster antimicrobial susceptibility testing of blood samples.

To obviate and/or mitigate at least some of these deficiencies in conventional methods of blood sample analysis, the present inventors have developed a method of performing antimicrobial susceptibly testing on blood cultures without the need to wait for the TTP or identification of a microorganism in the blood cultures.

As further described herein, the present inventors have developed a method for determining the antimicrobial susceptibility of a microorganism in a blood sample. This method may include incubating a first blood culture derived from the blood sample. The method may further include terminating the incubation of the first blood culture after some predetermined incubation time. In many cases, this results in a pre-positive culture—i.e., a culture which does not test positive for a microorganism using current conventional testing. In certain preferred embodiments the incubation of the first blood culture may be terminated up to about 20 hours or up to about 15 hours, or up to about 12 hours, or up to about 10 hours, or up to about 8 hours, or up to about 6 hours, or up to about 3 hours, after commencement of incubation to produce an incubated culture. In certain embodiments, the incubation may be terminated when the incubated culture achieves a bacterial concentration of at least about 1×10⁴ colony-forming units per milliliter (CFU/mL), about 5×10⁴ CFU/mL, about 1×10⁵ CFU/mL, or about 5×10⁵ CFU/mL. The method may further include performing a molecular phenotypic antimicrobial susceptibility test (AST) assay on the incubated culture to determine the antimicrobial susceptibility of a microorganism.

As further described herein, the present inventors have developed a method for determining the antimicrobial susceptibility of a microorganism in a blood sample, wherein prior to incubating a first blood culture of a blood sample, the method may further comprise the steps of: collecting a blood sample from a patient and transporting the blood sample to a testing facility.

In certain preferred embodiments, the total time between the step of collecting the blood sample and completing a molecular phenotypic antimicrobial susceptibility test (AST) assay on an incubated culture of a first blood culture may be less than about 20 hours or preferably less than about 10 hours, or more preferably less than about 8 hours.

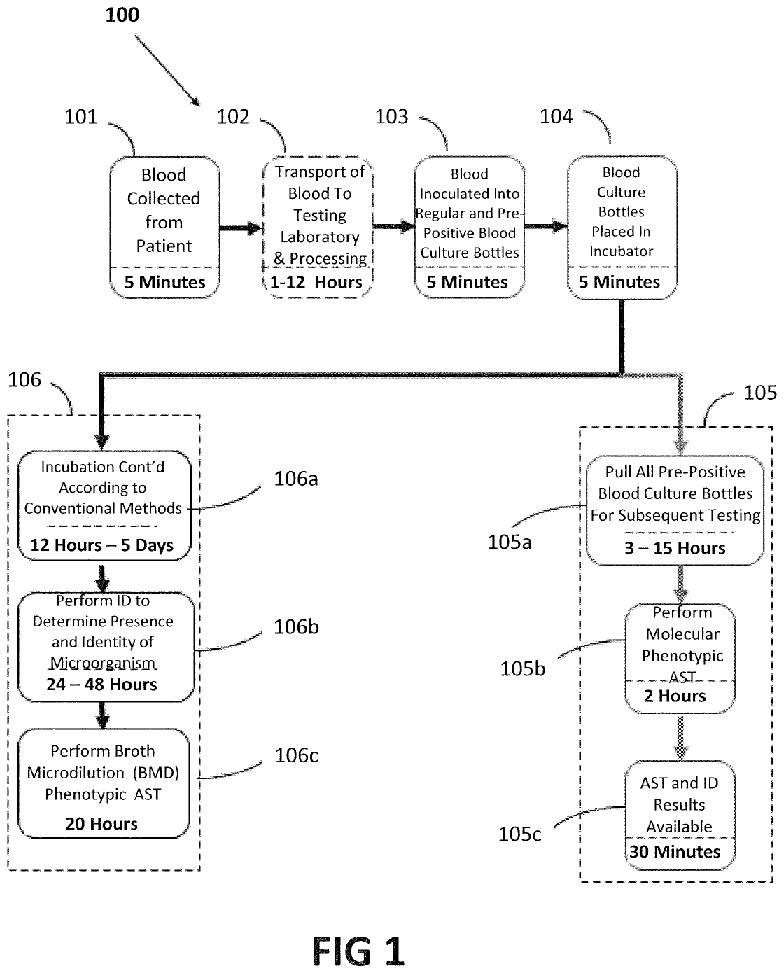

In accordance with one aspect of the teachings described herein, a method for determining the susceptibility of a microorganism in a blood sample obtained from a patient to an antimicrobial agent is described. FIG. 1 is a flowchart illustrating a preferred embodiment of this method.

Referring to FIG. 1, one example of a method 100 of determining the susceptibility of a microorganism in a blood sample includes a first step 101 of collecting a blood sample from a patient. In most embodiments of the method, the blood sample is believed to contain at least one microorganism or microbial species in a clinically relevant amount and may be suspected of containing two or more microorganisms of microbial species in a clinically relevant amount. The collection of a blood sample from a patient may take about 5 minutes.

Once the blood sample is collected from the patient, the method 100 may include a second step 102 of transporting the blood sample to a testing laboratory and processing of the blood sample. By way of non-limiting example, the processing of a blood sample may include sample labelling and organization, testing set up, etc. The transportation and processing of the blood sample may take from about 1 to 12 hours. Once the blood sample has been transported to a testing laboratory and processed, the method 100 may include the third step 103 of inoculating the blood sample into one or more blood culture containers.

As shown in FIG. 1, in certain preferred embodiments step 103 may include inoculating the blood sample into two different cultures: (1) a first blood culture; and (2) a second reference blood culture. By way of non-limiting example, the first blood culture may be configured to be used/analyzed in a pre-positive work path (such as those described herein), while the second reference blood culture may be configured to be used/analyzed in a conventional positive blood work path. The inoculation of the blood sample into one or more blood cultures may take around about 5 minutes. Once the blood sample has been inoculated into one or more blood cultures, the method 100 may include a fourth step 104 of incubating the blood cultures.

### Incubation of the Blood Culture

In certain preferred embodiments, the incubation step of the blood culture may comprise:
(a) disposing a test portion of the blood culture in a first incubation chamber, wherein the incubation chamber comprises (i) a first wall, (ii) a second wall opposed to the first wall, and (iii) at least one sidewall interconnecting the first wall and the second wall to define a chamber interior having a chamber volume and configured to contain a liquid, wherein a ratio of the first wall surface area to chamber volume is at least about 19 m⁻¹, wherein at least a portion of at least one of the first wall and second wall is gas permeable; and
(b) mixing the test portion of the blood culture by oscillating the incubation chamber back and forth along an oscillation path at a predetermined oscillation frequency.

In certain preferred embodiments, the incubation step of the blood culture may further comprise:
(c) disposing a control portion of the blood culture in a second incubation chamber, wherein the incubation chamber comprises (i) a first wall, (ii) a second wall opposed to the first wall, and (iii) at least one sidewall interconnecting the first wall and the second wall to define a chamber interior having a chamber volume and configured to contain a liquid, wherein a ratio of the first wall surface area to chamber volume is at least about 19 m⁻¹, wherein at least a portion of at least one of the first wall and second wall is gas permeable; and
(d) mixing the control portion of the blood culture by oscillating the incubation chamber back and forth along an oscillation path at a predetermined oscillation frequency.

In certain embodiments, the oscillation path may be an arcuate path with an oscillation angle of between about 100 and about 260 degrees. The predetermined oscillation frequency may be between about 1 and 5 Hz. The incubation chamber may be oscillated at an angular acceleration in a range between 100 to 500 rad/s².

In certain preferred embodiments, the blood culture when disposed in the first incubation chamber may occupy no more than ⅔ of the chamber volume, such that there remains a head space within the incubation chamber.

In some embodiments, the medium into which the test and/or control portions of the blood culture is disposed may be a container. In some embodiments, the container may be selected from the group of a tissue culture plate, vial, flask, microcentrifuge tube and centrifugal disk. In some embodiments, the container may be a well of a tissue culture plate. In some embodiments, the tissue culture plate may contain a plurality of wells (i.e., any number of wells). In some embodiments, the tissue culture plate may contain 6, 12, 24, 48, 96, or more wells. In some embodiments, the container may be a chamber of a centrifugal disc.

Optionally, multiple test chambers can be included in a single centrifugal disc, such that more than one test can be conducted using a common apparatus, but preferably in fluid isolation from each other.

In certain preferred embodiments, the incubation of the test portion of the blood culture may be done within a test incubation chamber on a centrifugal disc, and incubation of the control portion of the blood culture may be done within a control incubation chamber on the same centrifugal disc.

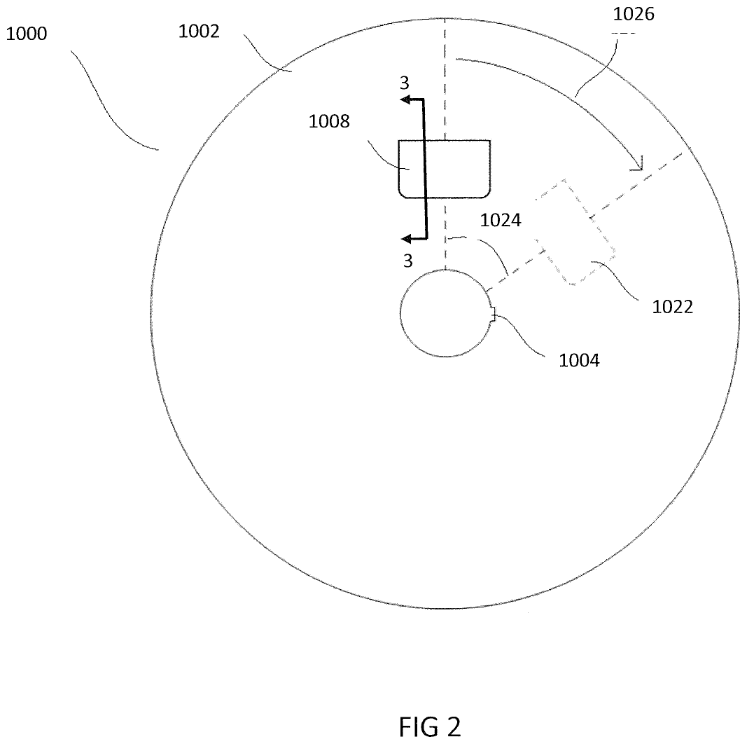

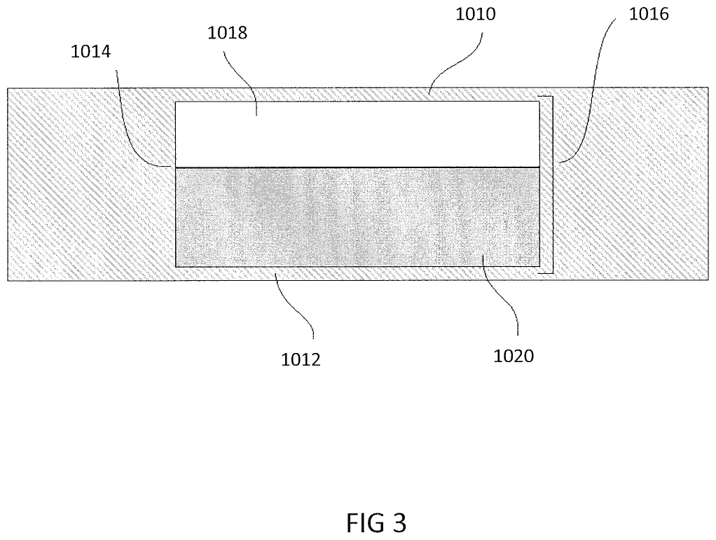

Referring to FIGS. 2 and 3, one example of centrifugal disc 1000 is schematically illustrated, having a body 1002 (e.g., a disc) with a mounting portion 1004 that is configured to be mounted to a suitable driving/spinning apparatus and an incubation chamber 1008. The incubation chamber 1008 in this example has an interior 1016 that is bounded by a first or upper wall 1010 (as illustrated in FIG. 3), an opposing second or lower wall 1012 and a sidewall 1014 extending therebetween. Preferably, at least one of the upper wall 1010 and lower wall 1012 can be formed from a material that is gas permeable.

Preferably, the incubation chamber 1008 is sized so that when a desired quantity of a sample 1020 (e.g. blood culture) is disposed in the interior 1016 it occupies nor more than ⅔ of the volume of the chamber 1008, whereby a head space 1018 to accommodate a cover gas/air remains within the interior 1016 of the chamber 1008.

To help facilitate incubation of the sample 1020, the disc 1000 may be oscillated, by a suitable device, along an oscillation path 1026 and by a suitable oscillation angle 1024 (which may be between about 100 and about 260 degrees) between an initiation position and a second position (indicated by dashed lines and reference character 1022).

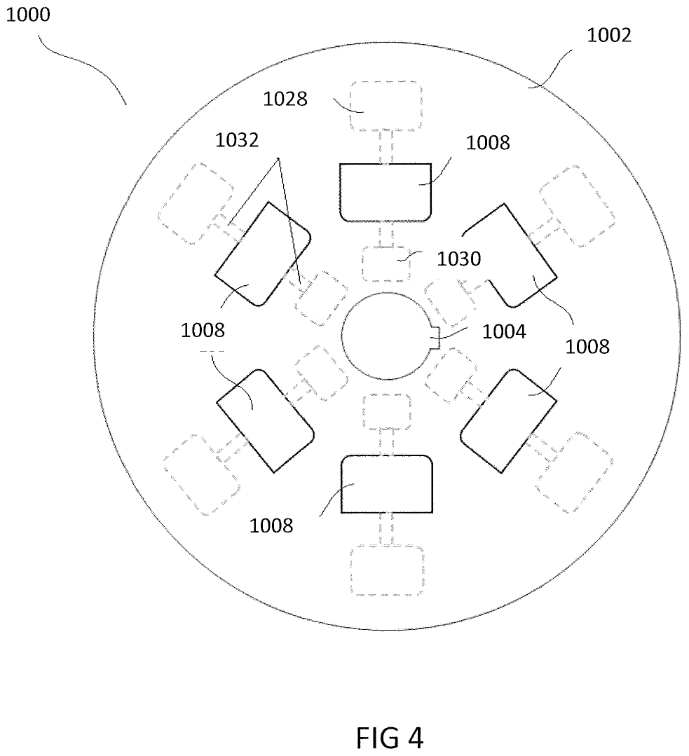

Optionally, as shown in FIG. 4, the disc 1000 may include multiple incubation chambers 1008 that spaced apart from, and fluidly isolated from each other. This may allow multiple samples to be tested simultaneously on a common body 1002. The body 1002 may also include other process/test chambers and fluid connections (illustrated via reference characters 1028, 1030 and 1032) that can accommodate other desired processes (lysing, rinsing, etc.).

The incubation step of the pre-positive blood culture may include those methods disclosed in International Application No. PCT/US2018/048906, which is incorporated herein by reference.

### Incubation of Reference Blood Culture

The reference blood culture may be incubated using conventional clinical microbiology testing methods and testing systems (e.g., Bactec™ by Becton Dickinson). Once the blood sample has been added to the blood culture bottle, the bottle is placed in the incubator, which heats the bottle at 37° C. with gentle, rocking agitation along the length of the cylindrical shape. The blood culture bottle contains a pH-sensitive resin that changes color when the media is acidified by the CO₂ that the microbes produce. When enough microbial growth has occurred, and enough CO₂ has been produced, the resin undergoes the required color change. This color change in the individual bottle is detected by the incubator, which then flags the bottle as positive for downstream testing.

### Post-Incubation Antibiotic Susceptibility Testing

As shown in FIG. 1, once the blood cultures have begun incubation, the method 100 may include subjecting the incubated blood cultures to one or more prescribed work paths (shown as steps 105 and 106 in FIG. 1). By way of non-limiting example, the first blood culture may be subjected to a pre-positive work path 105 and the second reference blood culture may be subjected to a conventional blood culture work path 106.

In certain preferred embodiments, the first blood culture may be subjected to a pre-positive work path 105 at or around the same time that the second reference blood culture may be subjected to a conventional blood culture work path 106.

### Termination of Incubation

As shown in FIG. 1, the pre-positive work path of method 100 may include terminating the incubation of the first blood culture at a pre-determined time. In most case this will create a pre-positive culture (step 105a). In certain preferred embodiments, this pre-determined time may be less than the average TTP that would be required to generate a detectable positive indication of a gram-positive organism in a blood sample. In other embodiments, this pre-determined time may be less than the average TTP that would be required to generate a detectable positive indication of a gram-negative organism in a blood sample.

By way of non-limiting example, the pre-determined incubation time to create a pre-positive culture may be up to around about 20 hours, or up to around about 15 hours, or up to around about 12 hours, or up to around about 10 hours, or up to around about 8 hours, or up to around about 6 hours, or up to around about 3 hours. As further shown in FIG. 1, method 100 may further include performing a molecular phenotypic antimicrobial susceptibility test (AST) assay on the incubated culture to determine the antimicrobial susceptibility of a microorganism in the blood sample (step 105b).

In certain preferred embodiments, the molecular phenotypic antimicrobial susceptibility test (AST) assay on the incubated culture (step 105b) may comprise:
(i) inoculating a test portion of the pre-positive culture in a medium containing a predetermined concentration of an antimicrobial agent;
(ii) inoculating a control portion of the pre-positive culture in a medium that does not contain the antimicrobial agent;
(iii) incubating the test portion and the control portion for an incubation period that is less than about 420 minutes;
(iv) determining a quantity of a nucleic acid molecule in the test portion and a quantity of the nucleic acid molecule in the control portion at the conclusion of the incubation period; and
(v) determining a susceptibility of the microorganism to the antimicrobial agent by comparing the quantity of the nucleic acid molecule in the test portion to the quantity of the nucleic acid molecule in the control portion.

In certain preferred embodiments, the molecular phenotypic antimicrobial susceptibility test (AST) assay (step 105b) may be performed on a plurality of test portions of the incubated cultures in parallel, in order to independently test the susceptibility of the incubated culture contemporaneously to a series of different antimicrobial agents.

Following the AST assay of step 105b, antimicrobial susceptibility and identification results may be obtained in about around 30 minutes (step 105c).

In some embodiments, the microorganism may be susceptible to the antimicrobial agent if the quantity of nucleic acid molecules of the microorganism in the antimicrobial agent-free inoculate is significantly more than the quantity of nucleic acid molecules of the microorganism in an inoculate comprising the microorganism and the antimicrobial agent. In some embodiments, the microorganism is not susceptible to the antimicrobial agent if the quantity of nucleic acid molecules of the microorganism in the antimicrobial agent-free inoculate is nearly equal, equal, or less than the quantity of nucleic acid molecules of the microorganism in an inoculate comprising the microorganism and the antimicrobial agent.

In certain preferred embodiments, the nucleic acid molecule may comprise at least one of deoxyribonucleic acid (DNA) and ribonucleic acid (RNA). By way of non-limiting example, the nucleic acid molecule may comprise ribosomal RNA (rRNA), pre-ribosomal RNA (prRNA), and/or mature RNA. More preferably, the nucleic acid molecule may comprise 16S rRNA and/or 23S rRNA.

In certain preferred embodiments, the antimicrobial agent may comprise at least one antibiotic agent. By way of non-limiting example, the antibiotic agent may be selected from the group consisting of gentamicin, ciprofloxacin, cefazolin, ceftriaxone, cefepime, ampicillin, trimethoprim-sulfamethoxazole, nitrofurantoin, fosfomycin, amoxicillin-clavulanate, amikacin, ertapenem, meropenem, tobramycin, levofloxacin, ceftazidime, ceftazidime-avibactam, piperacillin-tazobactam, colistin, cefoxitin, daptomycin, erythromycin, penicillin, oxacillin, linezolid, rifampin, tigecycline, quinupristin-daftopristin, vancomycin, and any combinations of two or more of these.

Determining the quantity of a nucleic acid molecule in the test portion and a quantity of the nucleic acid molecule in the control portion may be done using any suitable method, including those methods described in International Application No. PCT/US2018/047075, which is incorporated herein by reference. By way of non-limiting example, one example of a suitable method for determining the quantity of a nucleic acid molecule may include the steps of: (i) lysis to release rRNA; (ii) neutralization; (iii) hybridization of target rRNA with a capture probe and detector probe; and (iv) detection of capture probe—target rRNA—detector probe complexes. Each of these steps (i) through (iv) may be performed on the test portion and/or the control portion of the incubated test portion and the control portion. Optionally, the method of determining the quantity of a nucleic acid molecule may be performed at least partially, and preferably completely, automatically using a suitable apparatus.

In certain preferred embodiments, a chemical lysis to release rRNA may include:
(a) subjecting the test portion to mechanical lysis to cause disruption of a cellular membrane in the bacteria;
(b) contacting the test portion with an alkaline material to produce a lysate composition comprising the RNA; and
(c) recovering the lysate composition from the test portion.

Optionally, the lysing step may include at least one of chemical lysing, mechanical lysing and/or a combination thereof. In some embodiments, lysis may include both chemical and mechanical lysing operations. One example of a suitable lysing technique is described in International Application No. PCT/US2018/045211, which is incorporated herein by reference.

The neutralization step can be performed using any suitable method.

In some embodiments, the neutralization and hybridization steps may be conducted using universal probes. In other embodiments, the neutralization and hybridization steps may be conducted using species specific probes. By way of non-limiting examples, species-specific probes may include Escherichia coli, Klebsiella pneumoniae, Proteus mirabilis, Pseudomonas aeruginosa, Staphylococcus aureus, or Streptococcus agalactiae. By using species-specific probes, the signal of rRNA from different types of bacteria in mixed blood samples may be individually observed/counted and/or only signals from the desired, targeted bacteria may be counted. This may help facilitate the quantification of two or more different target bacteria within a common blood sample and may allow the concentrations of two or more target bacterial rRNA concentrations to be measured generally contemporaneously.

### Blood Culture Work Path

As shown in FIG. 1, the conventional blood culture work path 106 of method 100 may include continuing incubation of the reference blood culture according to conventional positive blood culture testing protocol (step 106a). The conventional blood culture work path of method 100 may further include performing a bacterial identification test (ID) on the second blood culture to determine if the reference blood culture is positive for a microorganism and the identity of the microorganism step (step 106b).

Techniques to determine if the reference blood culture is positive for a microorganism following incubation may, for example, include isolating the incubated reference blood culture on agar plates, gram staining the incubated reference blood culture, and carrying out a bacterial identification assay. If the incubated reference blood culture tests positive for the microorganism, the antibiotic susceptibility of the microorganism may be detected by performing a phenotypic antimicrobial susceptibility test (AST) assay on the incubated reference blood culture (step 106c) using broth microdilution (manual or automated). The ID and AST of the second reference blood culture may serve as a comparator or a control to verify the results of the AST performed on the pre-positive culture.

### Method of Treating Bloodstream Infections

The present inventors have further developed a method for treating a bloodstream infection in a patient using the antibiotic susceptibility testing methods disclosed herein. In certain embodiments, a method for treating a bloodstream infection in a patient may include:
(a) obtaining a blood sample from a patient;
(b) incubating a first blood culture derived from the blood sample;
(c) terminating the incubation of the first blood culture after pre-determined time from commencement of Step (b) to produce a pre-positive culture;
(d) performing a molecular phenotypic antimicrobial susceptibility test (AST) assay on the pre-positive culture determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

In certain preferred embodiments, this pre-determined time may be less than the average TTP that would be required to generate a detectable positive indication of a gram-positive organism in the blood sample. In other embodiments, this pre-determined time may be less than the average TTP that would be required to generate a detectable positive indication of a gram-negative organism in the blood sample. By way of non-limiting example, the pre-determined incubation time to create an incubated culture may be up to around about 20 hours, or up to around about 15 hours, or up to around about 12 hours, or up to around about 10 hours, or up to around about 8 hours, or up to around about 6 hours, or up to around about 3 hours, after commencement of incubation of the blood culture. In certain embodiments, the incubation may be terminated when the incubated culture achieves a bacterial concentration of at least about 1×10⁴ colony-forming units per milliliter (CFU/mL), about 5×10⁴ CFU/mL, about 1×10⁵ CFU/mL, or about 5×10⁵ CFU/mL.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

What is claimed is:

1. A method for determining an antimicrobial susceptibility of a microorganism in a blood sample, the method comprising the steps of:
(a) incubating a first blood culture derived from the blood sample;
(b) terminating the incubation of the first blood culture after a period of up to about 20 hours from commencement of Step (a) to produce an incubated culture; and
(c) performing a molecular phenotypic antimicrobial susceptibility test (AST) assay on the incubated culture to determine the antimicrobial susceptibility of a microorganism.

2. A method for determining an antimicrobial susceptibility of a microorganism in a blood sample, the method comprising the steps of:
(a) incubating a first blood culture derived from the blood sample;
(b) terminating the incubation of the first blood culture after a period of up to about 15 hours from commencement of Step (a) to produce an incubated culture; and
(c) performing a molecular phenotypic antimicrobial susceptibility test (AST) assay on the incubated culture to determine the antimicrobial susceptibility of a microorganism.

3. A method for determining an antimicrobial susceptibility of a microorganism in a blood sample, the method comprising the steps of:
(a) incubating a first blood culture derived from the blood sample;
(b) terminating the incubation of the first blood culture after a period of up to about 12 hours from commencement of Step (a) to produce an incubated culture; and
(c) performing a molecular phenotypic antimicrobial susceptibility test (AST) assay on the incubated culture to determine the antimicrobial susceptibility of a microorganism.

4. A method for determining an antimicrobial susceptibility of a microorganism in a blood sample, the method comprising the steps of:
(a) incubating a first blood culture derived from the blood sample;
(b) terminating the incubation of the first blood culture after a period of up to about 10 hours from commencement of Step (a) to produce an incubated culture; and
(c) performing a molecular phenotypic antimicrobial susceptibility test (AST) assay on the incubated culture to determine the antimicrobial susceptibility of a microorganism.

5. A method for determining an antimicrobial susceptibility of a microorganism in a blood sample, the method comprising the steps of:
(a) incubating a first blood culture derived from the blood sample;
(b) terminating the incubation of the first blood culture after a period of up to about 8 hours from commencement of Step (a) to produce an incubated culture; and
(c) performing a molecular phenotypic antimicrobial susceptibility test (AST) assay on the incubated culture to determine the antimicrobial susceptibility of a microorganism.

6. A method for determining an antimicrobial susceptibility of a microorganism in a blood sample, the method comprising the steps of:
(a) incubating a first blood culture derived from the blood sample;
(b) terminating the incubation of the first blood culture after a period of up to about 6 hours from commencement of Step (a) to produce an incubated culture; and
(c) performing a molecular phenotypic antimicrobial susceptibility test (AST) assay on the incubated culture to determine the antimicrobial susceptibility of a microorganism.

7. A method for determining an antimicrobial susceptibility of a microorganism in a blood sample, the method comprising the steps of:
(a) incubating a first blood culture derived from the blood sample;
(b) terminating the incubation of the first blood culture after a period of up to about 3 hours from commencement of Step (a) to produce an incubated culture; and
(c) performing a molecular phenotypic antimicrobial susceptibility test (AST) assay on the incubated culture to determine the antimicrobial susceptibility of a microorganism.

8. The method defined in any one of claims 1 to 7, where the incubated culture has a bacterial concentration of at least about 1×10⁴ colony-forming units per milliliter (CFU/mL).

9. The method defined in any one of claims 1 to 7, where the incubated culture has a bacterial concentration of at least about 5×10⁴ colony-forming units per milliliter (CFU/mL).

10. The method defined in any one of claims 1 to 7, where the incubated culture has a bacterial concentration of at least about 1×10⁵ colony-forming units per milliliter (CFU/mL).

11. The method defined in any one of claims 1 to 7, where the incubated culture has a bacterial concentration of at least about 5×10⁵ colony-forming units per milliliter (CFU/mL).

12. A method for determining an antimicrobial susceptibility of a microorganism in a blood sample, the method comprising the steps of:
(a) incubating a first blood culture derived from the blood sample;
(b) terminating the incubation of the first blood culture when a bacterial concentration of about 1×10⁴ colony-forming units per milliliter (CFU/mL) is reached to produce an incubated culture; and
(c) performing a molecular phenotypic antimicrobial susceptibility test (AST) assay on the incubated culture to determine the antimicrobial susceptibility of a microorganism.

13. A method for determining an antimicrobial susceptibility of a microorganism in a blood sample, the method comprising the steps of:
(a) incubating a first blood culture derived from the blood sample;
(b) terminating the incubation of the first blood culture when a bacterial concentration of about 5×10⁴ colony-forming units per milliliter (CFU/mL) is reached to produce an incubated culture; and
(c) performing a molecular phenotypic antimicrobial susceptibility test (AST) assay on the incubated culture to determine the antimicrobial susceptibility of a microorganism.

14. A method for determining an antimicrobial susceptibility of a microorganism in a blood sample, the method comprising the steps of:
(a) incubating a first blood culture derived from the blood sample;
(b) terminating the incubation of the first blood culture when a bacterial concentration of about 1×10⁵ colony-forming units per milliliter (CFU/mL) is reached to produce an incubated culture; and
(c) performing a molecular phenotypic antimicrobial susceptibility test (AST) assay on the incubated culture to determine the antimicrobial susceptibility of a microorganism.

15. A method for determining an antimicrobial susceptibility of a microorganism in a blood sample, the method comprising the steps of:
(a) incubating a first blood culture derived from the blood sample;
(b) terminating the incubation of the first blood culture when a bacterial concentration of about 5×10⁵ colony-forming units per milliliter (CFU/mL) is reached to produce an incubated culture; and
(c) performing a molecular phenotypic antimicrobial susceptibility test (AST) assay on the incubated culture to determine the antimicrobial susceptibility of a microorganism.

16. The method defined in any one of claims 1 to 15, wherein Step (a) is conducted for a period of at least about 30 minutes.

17. The method defined in any one of claims 1 to 15, wherein Step (a) is conducted for a period of at least about 45 minutes.

18. The method defined in any one of claims 1 to 15, wherein Step (a) is conducted for a period of at least about 1 hour.

19. The method defined in any one of claims 1 to 15, wherein Step (a) is conducted for a period of at least about 2 hours.

20. The method defined in any one of claims 1 to 15, wherein Step (a) is conducted for a period of at least about 3 hours.

21. The method defined in any one of claims 1 to 15, wherein Step (a) is conducted for a period of at least about 4 hours.

22. The method defined in any one of claims 1 to 15, wherein Step (a) is conducted for a period of at least about 5 hours.

23. The method defined in any one of claims 1 to 22, wherein the Step (c) comprises the following steps:
(i) inoculating a test portion of the incubated culture in a medium containing a predetermined concentration of an antimicrobial agent;
(ii) inoculating a control portion of the incubated culture in a medium that does not contain the antimicrobial agent;
(iii) incubating the test portion and the control portion for an incubation period that is less than 420 minutes;
(iv) determining a quantity of a nucleic acid molecule in the test portion and a quantity of the nucleic acid molecule in the control portion at the conclusion of the incubation period; and
(v) determining susceptibility of the microorganism to the antimicrobial agent by comparing the quantity of the nucleic acid molecule in the test portion to the quantity of the nucleic acid molecule in the control portion.

24. The method defined in claim 23, wherein the nucleic acid molecule comprises at least one of deoxyribonucleic acid (DNA) and ribonucleic acid (RNA).

25. The method defined in claim 23, wherein the nucleic acid molecule comprises RNA.

26. The method defined in claim 23, wherein the nucleic acid molecule comprises ribosomal RNA (rRNA).

27. The method defined in claim 23, wherein the nucleic acid molecule comprises pre-ribosomal RNA (prRNA).

28. The method defined in claim 23, wherein the nucleic acid molecule comprises mature RNA.

29. The method defined in claim 23, wherein the nucleic acid molecule comprises at least one of 16S rRNA and 23S rRNA.

30. The method defined in any one of claims 23 to 29, wherein the antimicrobial agent comprises at least one antibiotic agent.

31. The method defined in claim 30, wherein the antibiotic agent comprises at least one of Gentamicin, Ciprofloxacin, Cefazolin, Ceftriaxone, Cefepime, Ampicillin, Trimethoprim-Sulfamethoxazole, Nitrofurantoin, Fosfomycin, Amoxicillin-Clavulanate, Amikacin, Ertapenem, Meropenem, Tobramycin, Levofloxacin, Ceftazidime, Ceftazidime-Avibactam, Piperacillin-Tazobactam, Colistin, Cefoxitin, Daptomycin, Erythromycin, Penicillin, Oxacillin, Linezolid, Rifampin, Tigecycline, Quinupristin-daftopristin, Vancomycin and combinations thereof.

32. The method defined in any one of claims 23 to 31, wherein the incubation period is equal to or less than 240 minutes.

33. The method defined in any one of claims 23 to 32, wherein the incubation of the test portion comprises:
disposing the test portion of the incubated culture in a first incubation chamber, wherein the incubation chamber comprises
(i) a first wall;
(ii) a second wall opposed to the first wall; and
(iii) at least one sidewall interconnecting the first wall and the second wall to define a chamber interior having a chamber volume and configured to contain a liquid, wherein a ratio of the first wall surface area to chamber volume is at least about 19 m-1, wherein at least a portion of at least one of the first wall and second wall is gas permeable; and
mixing the test portion of the incubated culture by oscillating the incubation chamber back and forth along an oscillation path at a predetermined oscillation frequency.

34. The method defined in claim 33, wherein the incubation of the control portion comprises:
disposing the control portion of the incubated culture in a second incubation chamber, wherein the incubation chamber comprises
(i) a first wall,
(ii) a second wall opposed to the first wall, and
(iii) at least one sidewall interconnecting the first wall and the second wall to define a chamber interior having a chamber volume and configured to contain a liquid, wherein a ratio of the first wall surface area to chamber volume is at least about 19 m-1, wherein at least a portion of at least one of the first wall and second wall is gas permeable; and
mixing the control portion of the incubated culture by oscillating the incubation chamber back and forth along an oscillation path at a predetermined oscillation frequency.

35. The method defined in claim 33 or 34, wherein the oscillation path is an arcuate path with an oscillation angle of between about 100 and about 260 degrees.

36. The method defined in claim 33 or 34, wherein the predetermined oscillation frequency is between 1 and 5 Hz.

37. The method defined in claim 33 or 34, wherein the incubation chamber is oscillated at an angular acceleration in a range between 100 to 500 rad/s².

38. The method defined in claim 33 or 34, wherein the incubated culture when disposed in the first incubation chamber occupies no more than ⅔ of the chamber volume, such that there remains a head space within the incubation chamber.

39. The method defined in any one of claims 23 to 38, wherein after incubating the test portion and the control portion of the incubated culture, lysis is performed on the test portion.

40. The method defined in claim 39, wherein the lysis of the test portion comprises:
(i) subjecting the test portion to mechanical lysis to cause disruption of a cellular membrane;
(ii) subjecting the test portion to chemical lysis to produce a lysate composition comprising the nucleic acid molecule; and
(iii) recovering the lysate composition from the test portion.

41. The method defined in claim 39, wherein the lysis of the test portion comprises:
(i) subjecting the test portion to chemical lysis to produce a lysate composition comprising the nucleic acid molecule; and
(ii) recovering the lysate composition from the test portion.

42. The method defined in claim 40 or 41, wherein the chemical lysis comprises:
contacting the test portion with an alkaline material to produce a lysate composition comprising the nucleic acid molecule.

43. The method defined in any one of claims 23 to 42, wherein after incubating the test portion and the control portion of the incubated culture, lysis is performed on the control portion.

44. The method defined in claim 43, wherein the lysis of the control portion comprises:
(i) subjecting the control portion to mechanical lysis to cause disruption of a cellular membrane;
(ii) subjecting the control portion to chemical lysis to produce a lysate composition comprising the nucleic acid molecule; and
(iii) recovering the lysate composition from the control portion.

45. The method defined in claim 43, wherein the lysis of the control portion comprises:
(i) subjecting the control portion to chemical lysis to produce a lysate composition comprising the nucleic acid molecule; and
(ii) recovering the lysate composition from the control portion.

46. The method defined in claim 44 or 45, wherein the chemical lysis comprises:
contacting the control portion with an alkaline material to produce a lysate composition comprising the nucleic acid molecule.

47. The method defined in any one of claims 1 to 46, comprising the further steps of:
(d) incubating a second blood culture derived from the blood sample until the second blood culture tests positive for the presence of a microorganism;
(e) performing a bacterial identification test (ID) on the second blood culture to determine the identity of the microorganism; and
(f) performing a phenotypic antimicrobial susceptibility test (AST) assay on the second blood culture to determine the antimicrobial susceptibility of the microorganism.

48. The method defined in claim 47, wherein the incubation of the first and second blood cultures are performed at or around the same time.

49. The method defined in claim 47 or 48, wherein the ID and AST performed on the second blood culture serves as a comparator to verify the results of the AST performed on the incubated culture.

50. The method defined in any of claims 1 to 49, wherein the molecular phenotypic antimicrobial susceptibility test (AST) assay of Step (c) is performed on a plurality of test portions of the incubated cultures in parallel, in order to test the susceptibility of the incubated culture to a plurality of antimicrobial agents simultaneously.

51. A method for treating a bloodstream infection in a patient, the method comprising the steps of:
(a) obtaining a blood sample from a patient;
(b) incubating a first blood culture derived from the blood sample;
(c) terminating the incubation of the first blood culture after a period of up to about 20 hours from commencement of Step (b) to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

52. A method for treating a bloodstream infection in a patient, the method comprising the steps of:
(a) obtaining a blood sample from a patient;
(b) incubating a first blood culture derived from the blood sample;
(c) terminating the incubation of the first blood culture after a period of up to about 15 hours from commencement of Step (b) to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

53. A method for treating a bloodstream infection in a patient, the method comprising the steps of:
(a) obtaining a blood sample from a patient;
(b) incubating a first blood culture derived from the blood sample;
(c) terminating the incubation of the first blood culture after a period of up to about 12 hours from commencement of Step (b) to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

54. A method for treating a bloodstream infection in a patient, the method comprising the steps of:
(a) obtaining a blood sample from a patient;
(b) incubating a first blood culture derived from the blood sample;
(c) terminating the incubation of the first blood culture after a period of up to about 10 hours from commencement of Step (b) to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

55. A method for treating a bloodstream infection in a patient, the method comprising the steps of:
(a) obtaining a blood sample from a patient;
(b) incubating a first blood culture derived from the blood sample;
(c) terminating the incubation of the first blood culture after a period of up to about 8 hours from commencement of Step (b) to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

56. A method for treating a bloodstream infection in a patient, the method comprising the steps of:
(a) obtaining a blood sample from a patient;
(b) incubating a first blood culture derived from the blood sample;
(c) terminating the incubation of the first blood culture after a period of up to about 6 hours from commencement of Step (b) to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

57. A method for treating a bloodstream infection in a patient, the method comprising the steps of:
(a) obtaining a blood sample from a patient;
(b) incubating a first blood culture derived from the blood sample;
(c) terminating the incubation of the first blood culture after a period of up to about 3 hours from commencement of Step (b) to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

58. The method defined in any one of claims 51 to 57, where the incubated culture has a bacterial concentration of at least about 1×10⁴ colony-forming units per milliliter (CFU/mL).

59. The method defined in any one of claims 51 to 57, where the incubated culture has a bacterial concentration of at least about 5×10⁴ colony-forming units per milliliter (CFU/mL).

60. The method defined in any one of claims 51 to 57, where the incubated culture has a bacterial concentration of at least about 1×10⁵ colony-forming units per milliliter (CFU/mL).

61. The method defined in any one of claims 51 to 57, where the incubated culture has a bacterial concentration of at least about 5×10⁵ colony-forming units per milliliter (CFU/mL).

62. A method for treating a bloodstream infection in a patient, the method comprising the steps of:
(a) obtaining a blood sample from a patient;
(b) incubating a first blood culture derived from the blood sample;
(c) terminating the incubation of the first blood culture when a bacterial concentration of about 1×10⁴ colony-forming units per milliliter (CFU/mL) is reached to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

63. A method for treating a bloodstream infection in a patient, the method comprising the steps of:
(a) obtaining a blood sample from a patient;
(b) incubating a first blood culture derived from the blood sample;
(c) terminating the incubation of the first blood culture when a bacterial concentration of about 5×10⁴ colony-forming units per milliliter (CFU/mL) is reached to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

64. A method for treating a bloodstream infection in a patient, the method comprising the steps of:
(a) obtaining a blood sample from a patient;
(b) incubating a first blood culture derived from the blood sample;
(c) terminating the incubation of the first blood culture when a bacterial concentration of about 1×10⁵ colony-forming units per milliliter (CFU/mL) is reached to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

65. A method for treating a bloodstream infection in a patient, the method comprising the steps of:
(a) obtaining a blood sample from a patient;
(b) incubating a first blood culture derived from the blood sample;
(c) terminating the incubation of the first blood culture when a bacterial concentration of about 5×10⁵ colony-forming units per milliliter (CFU/mL) is reached to produce an incubated culture;
(d) performing a molecular phenotypic antimicrobial susceptibility test (AST) assay on the incubated culture to determine the antimicrobial susceptibility of a microorganism in the blood sample;
(e) identifying at least one antimicrobial agent to which the microorganism in the blood sample is susceptible; and
(f) administering the identified antimicrobial agent to the patient to treat the bloodstream infection.

66. The method defined in any one of claims 51 to 65, comprising the subject matter of any one of claims 16 to 50.
